(19) United States
(12) Patent Application Publication
(10) Pub. No.: US 2023/0414153 A1
(43) Pub. Date: Dec. 28, 2023

(54) Methods and Apparatuses for Determining a QT Interval of an ECG Signal

(71) Applicant: BIOTRONIK SE & Co. KG, Berlin (DE)

(72) Inventors: Swetha Vennelaganti, King City, OR (US); Ravi Kiran Kondama Reddy, Portland, OR (US); R. Hollis Whittington, Portland, OR (US)

(73) Assignee: BIOTRONIK SE & Co. KG, Berlin (DE)

(21) Appl. No.: 18/252,731

(22) PCT Filed: Nov. 22, 2021
(86) PCT No.: PCT/EP2021/082519

§ 371 Date: May 12, 2023

Related U.S. Application Data
(60) Provisional application No. 63/118,172, filed on Nov. 25, 2020.

(30) Foreign Application Priority Data
Jan. 14, 2021 (EP) 21151519.2

(51) Int. Cl. A61B 5/36

(52) U.S. Cl. CPC A61B 5/36

(57) ABSTRACT

The disclosure relates to a method for determining a QT interval of an electrocardiogram (ECG) signal comprising the steps of: determining a QT interval of an electrocardiogram (ECG) signal comprising the steps of: determining a maximum slope of the ECG signal after a T wave maximum of the ECG signal, and fixing an end of the QT interval by performing, for example, extrapolating the maximum slope to a zero line of the ECG signal.

### CROSS-REFERENCE TO RELATED APPLICATIONS

This application is the United States National Phase under 35 U.S.C. § 371 of PCT International Patent Application No. PCT/EP2021/082519, filed on Nov. 22, 2021, which claims the benefit of European Patent Application No. 21151519.2, filed on Jan. 14, 2021, and U.S. Provisional Patent Application No. 63/118,172, filed on Nov. 25, 2020, the disclosures of which are hereby incorporated by reference herein in their entireties.

### TECHNICAL FIELD

The present disclosure relates to methods and apparatuses for determining a QT interval of an electrocardiogram (ECG) signal. In particular, the present disclosure relates to methods and apparatuses for automatically determining QT intervals of ECG signals (that may be required for providing important diagnostic parameters) and for long-term monitoring of patients.

### BACKGROUND

Monitoring of electrocardiogram (ECG) signals, e.g., the heart's electrical signals, is, for example, used for monitoring the cardiac cycles of patients in order to detect various disorders or abnormalities. Typically, an electrocardiogram (ECG) has five distinct waves labeled as P, Q, R, S and T waves occurring in this sequence in a normal cardiac cycle. The Q, R, and S waves are also called QRS complex. The signal associated with the QRS complex is typically larger than that of the P wave and the signal represented by the T wave.

On an ECG, the ventricular depolarization and the subsequent repolarization is represented by the duration between the beginning of the Q wave and the end of the T wave, which is also called the QT interval. The QT interval can vary within a person depending on the heart rate. Hence, it is often corrected for the heart rate and is denoted as QTc, which can then provide valuable diagnostic information. Abnormally long or short QTc intervals can be congenital, or drug-induced and are associated with an increased risk of life-threatening ventricular arrhythmias and sudden cardiac deaths (SCD).

The drug-induced long QT syndrome (LQTS) can be a result of using anti-arrhythmic or similar medication, putting vulnerable population at risk. A diagnostic tool to monitor and titrate drug therapy related changes to the QTc interval can help mitigate the risk of this syndrome.

In patients with an acute ischemic stroke, the syndrome of paroxysmal atrial fibrillation (PAF) often goes undetected. A prolongation of the QTc interval is potentially a strong predictor for poststroke PAF. Long term monitoring of the QTc interval can be helpful for better management of patient's overall health.

A device that can periodically monitor a patient's QTc interval over a long period either from the surface of the body or from within the body may allow mitigating the above risks and help with disease management.

A QT detection algorithm designed for continuous long-term monitoring using an insertable cardiac monitor (ICM) is described earlier. R waves are sensed by using a dual sensing scheme. Inter alia, RR intervals are computed, and based on the RR interval, QT algorithm parameters are computed.

The patent application U.S. Publication No. 2018/0256060 A1 describes a system and method of automatically monitoring QT intervals in a patient based on one or more ECG signals received from attached monitoring devices. Each ECG signal is analyzed to detect attributes of the first and second ECG signals, including QRS onset information, QRS peak information, and T-wave offset information. A QT interval is calculated based on QRS onset information derived from the first ECG signal and T-wave offset information derived from the second ECG signal. The calculated QT interval is compared to thresholds to detect elongation of the QT interval and an alert is generated in response to a detected elongated QT interval.

However, the algorithms of the above document are less than optimal. For example, they use ECG templates in order to determine the QT interval. However, the accuracy of the QT interval determination using this scheme is reduced when the heart rate significantly deviates from the ECG template heart rate. The requirement of similar heart rate and ECG template rate limits the QT interval determination to a variation range of the heart rate of only five beats per minute (bpm).

Further, various ECG signal acquisition tools may have an offset associated with the ECG signal recording. It is assumed in the prior art that an ICM can remove any baseline offset in the ECG signal that directly impacts an isoelectric baseline. Typically, the applied algorithms perform well when the isoelectric baseline matches the zero line of the ECG voltage signal. But they do not perform well when there is a large offset and/or a drift in the ECG signal with respect to the zero line of the ECG voltage signal. Potentially, in an ICM and/or in a QT or in a QTc measurement system, an ECG signal can have a significant offset to the zero line of the ECG signal. This offset may result in an incorrect determination of the length of the QT interval. Further, a drift of the ECG signal may lead to a fluctuation of the QT interval length which is not caused by the patient's heartbeat.

The present disclosure is directed toward overcoming one or more of the above-mentioned problems, though not necessarily limited to embodiments that do.

### SUMMARY

There is therefore a need to provide methods and apparatuses for improving the determination of the QT interval of an electrocardiogram (ECG) signal which at least partially avoid the drawbacks discussed above. In particular, it may be considered as an object to provide methods and apparatuses for optimizing the determination of an end of a T wave of the ECG signal.

In some implementations, the present disclosure provides methods and apparatuses for determining a QT interval of an electrocardiogram (ECG) signal.

According to a first exemplary embodiment, a method for determining a QT interval of an electrocardiogram (ECG) signal comprises the steps of: (a) determining a maximum slope of the ECG signal after a T wave maximum of the ECG signal, and (b) fixing an end of the QT interval at least in part based on performing at least one of:
extrapolating the maximum slope to a zero line of the ECG signal;
determining a time position of the ECG signal after the T wave maximum at which the slope is for the first time within a range of 70% to 20%, preferred 66% to 25% of the maximum slope;
determining a time position at which an absolute value of a difference of the ECG signal and a product of the maximum slope multiplied by a length of a time interval which is used for determining the maximum slope is below a predetermined threshold;
calculating a second derivative of the ECG signal to determine an inflection point which is closest to the time position of the maximum slope. For example, the end of the QT interval may be fixed by performing at least one of the above items.

According to another, second, exemplary embodiment, a method for determining a QT interval of an electrocardiogram (ECG) signal comprises the steps of: (a) searching for a zero slope of the ECG signal after a T wave maximum of the ECG signal; and (b) fixing an end of the QT interval of the ECG signal at least in part based on determining a time position of a first zero slope of the ECG signal. For example, the end of the QT interval may be fixed by determining a time position of a first zero slope of the ECG signal.

A zero slope of the ECG signal can define an isoelectric line of the ECG signal, and may thus indicate the end of the T wave. The isoelectric line and/or a zero-voltage line (of an ICM) may be used as a zero line of the ECG signal for extrapolating the maximum slope to the zero line. However, the isoelectric line and the zero-voltage line of an ECG signal do not necessarily have the same electrical potential. Rather, the isoelectric line may have an offset and/or a drift with respect to the zero line of the ICM. Therefore, extrapolating the maximum slope to the zero line or the isoelectric line may result in different endpoints of the QT interval. Some of the methods or the algorithms described herein for determining the end of the QT interval may depend on this offset which may therefore be removed for an accurate determination of the QT interval length. Therefore, it is important to precisely fix the isoelectric baseline with respect to the zero line and to remove any offset of drift or the ECG signal.

In a further exemplary embodiment, a method for determining a QT interval of an electrocardiogram (ECG) signal comprises the steps of determining a maximum slope of the ECG signal after a T wave maximum of the ECG signal, and extrapolating the maximum slope to a zero line of the ECG signal for fixing an end of the QT interval. For example, the end of the QT interval may be fixed as an intersection of the extrapolation of the maximum slope and the zero line. The zero line can, for example, be a zero voltage line or an isoelectric line.

According to other aspects, methods and algorithms are provided for determining the end or the endpoint of the T wave which do not refer to the zero-voltage line. These may be less sensitive to an offset or drift of the ECG signal with respect to the zero line.

Further, depending on the specific form of the ECG signal, a specific approach may be used for determining the end of the T wave. For example, the approaches referring to the isoelectric line and/or the zero line may be used when the isoelectric line of the ECG signal matches the zero line of the ICM and/or a QT measurement system (e.g., to within a certain threshold). On the other hand, the approaches which do not rely on the isoelectric line and/or the zero line of the ECG signal for fixing the end of the T wave can, for example, be applied if the ECG signal has an offset and/or a drift regarding the zero line (e.g., above a certain threshold). Consequently, a comprehensive tool may be provided which can accurately determine the length of the QT interval for various forms of the ECG signal.

The change of slope methods along with/without the second differential method as described herein provide an accurate measurement tool for detecting an endpoint of the T wave. Thus, they may increase the performance of a QT measurement algorithm, thereby providing critical diagnostic information.

Moreover, it is a beneficial effect of the method of the second embodiment that the determination of neither the beginning nor the end of the QT interval refers to the zero line. Thus, this method is generally less susceptible to drifts of the ECG signal.

In another exemplary embodiment, a method for determining a QT interval of an electrocardiogram (ECG) signal comprises: searching for a zero slope of the ECG signal after a T wave maximum of the ECG signal; and fixing an end of the QT interval of the ECG signal by determining a time position of a first zero slope of the ECG signal.

According to another exemplary embodiment, a method for determining a QT interval of an electrocardiogram (ECG) signal comprises: searching for a zero slope of the ECG signal after a T wave maximum of the ECG signal for fixing an end of the QT interval of the ECG signal. In some examples, the zero slope may be a first zero slope, and the method may further include determining a time of the first zero slope for fixing an end of the QT interval of the ECG signal.

A zero slope of the ECG signal can define an isoelectric line of the ECG signal. The isoelectric line and/or a zero voltage line may be used as the zero line of the ECG signal for extrapolating the maximum slope to the zero line. The isoelectric line and the zero voltage line of an ECG signal do not necessarily have the same electrical potential.

In an aspect, the disclosed methods can enable an implantable cardiac monitor (ICM) and/or a wearable cardiac monitor (WCM) to automatically determine QT intervals and QT intervals which can be corrected with respect to the heart frequency (QTc). Thus, the methods can allow measuring the QT interval periodically and monitoring changes in the QT and/or the QTc interval over a long period. Therefore, they can, in some examples, enable establishing a useful diagnostic tool which can be an integral part of patient's health management. The methods can be designed to measure the QTc interval according to the current clinical guidelines.

Long term monitoring of the QT interval provides the diagnosis of the long QT syndrome (LQTS) and/or sudden cardiac death (SCD) risk mitigation. Further, the methods can allow the management of long-term QT interval trend calculation for drug monitoring and titration and the estimation of risk factors for paroxysmal atrial fibrillation (PAF) detection. The methods can also allow detecting aberrant heart rhythms and can enable the provision of systematic screening tools to the physicians and can become part of a patient's therapy management. The calculation of QT intervals in an implant can allow a very low data rate to be used to transmit this data (one QTc number, or a QT interval plus the heart rate, for example), rather than transmitting an entire ECG snapshot/strip. Also, this parameter, which is well known to electrophysiologists and cardiologists, can be more easily integrated into an electronic health record when the value/parameter is already known and does not need to be extracted.

Determining the QT interval may comprise using at least two items of feature b. of the first exemplary embodiment and averaging at least two results obtained when using the at least two items.

As discussed above, different items or approaches may be used for different forms of the ECG signal. But it is also possible apply two or more of the above indicated approaches in combination and their results may be averaged to improve the accuracy of the determination of the end of the T wave or the QT interval. The averaging of the results may be a linear averaging or a weighted averaging. The weights used for averaging may depend on the specific characteristics of the ECG signal.

Selecting one or more of the items or approaches of the above list may depend on a form of the ECG signal. Hence, the methods described herein enable a flexible adaptation of the QT interval length determination on varying ECG signals.

Moreover, it is also possible to combine one or more items of the first embodiment with the method of the second embodiment.

Determining a QT interval of an ECG signal can further comprise the step of determining a start of a Q wave of the ECG signal.

Determining the start of the Q wave can comprise at least one of: determining a sense event marker and determining a QRS complex.

The sense event marker can detect at least one of: a predetermined variation of the zero slope of the ECG signal in a PQ segment and an electrical potential exceeding a predetermined threshold in the PQ segment. Typically, the signal height and the steepness of the slopes of the ECG signal are larger in the QRS complex than during the T wave. Hence, the determination of the start of the Q wave can normally be determined with high precision. Or in other words, the accuracy of the determination of the QT interval length is mainly defined by the precision with which the end or the endpoint of the T wave can be fixed.

Another aspect further comprises the step of determining the QT interval as a difference between the end of the QT interval and the start of the Q wave.

The methods for determining a QT interval of an ECG signal can further comprise the step of determining a blanking period having a duration of the QRS complex and an ST segment of the ECG signal. The blanking period may be started after detecting a sense event marker or based on a QRS template.

In a further aspect, the duration of the blanking period comprises a period of 150 ms to 350 ms, preferably 200 ms to 300 ms, most preferably 230 ms to 270 ms.

The methods for determining a QT interval of an ECG signal can determine the start of the Q wave and the end of the T wave from a single cardiac cycle. It is also possible to determine the QT interval by averaging QT intervals of several cardiac cycles, in particular several cardiac cycles having a similar heart frequency.

Detecting the QRS complex can trigger a detection hold-off period (DHP). During the DHP no further detection events can be generated which effectively limits the maximum detection rate.

Determining the T wave maximum can comprise determining a T wave maximum search interval for detecting the T wave maximum.

The T wave maximum search interval can begin at the end of an ST segment of the ECG signal, and/or the T wave maximum search interval can have a duration of 150 ms to 600 ms, preferably 200 ms to 500 ms, and most preferably 250 ms to 400 ms.

The T wave maximum search interval can begin at the end of the blanking period. The T wave maximum search interval can begin at the start of the Q wave or at a predetermined time period after the start of the Q wave. The predetermined time period between the start of the Q wave and the beginning of the T wave maximum search interval may comprise a duration of 150 ms to 350 ms, preferably 200 ms to 300 ms, most preferably 230 ms to 270 ms.

Determining the T wave maximum can comprise searching for a maximum voltage level of the ECG signal in the T wave maximum search interval.

Determining the maximum slope can comprise searching of a maximum slope in the T wave maximum search interval after the T wave maximum.

Determining the maximum slope can comprise calculating a slope of the ECS signal by averaging the slope within a time interval of 2 ms to 100 ms, preferably 5 ms to 80 ms, more preferred 10 ms to 60 ms, and most preferred 20 to 40 ms.

Calculating the slope can comprise averaging 2 samples to 100 samples, preferably 4 samples to 80 samples, more preferred 8 samples to 60 samples, and most preferred 16 samples to 40 samples. Calculating the slope can comprise using a sampling frequency of 2² Hz to 2¹² Hz, preferably 2³ Hz to 2¹¹ Hz, more preferred 2⁴ Hz to 2¹⁰ Hz, most preferred 2⁵ Hz to 2⁹ Hz.

The methods for determining a QT interval of an ECG signal can comprise the step of filtering the ECG signal, wherein preferably filtering the ECG signal can comprise using at least one of: a high pass filter, a wavelet filter, and a median filter. Filtering the ECG signal comprise digitally filtering the ECG signal. The methods for determining a QT interval of an ECG signal can comprise the step of filtering the ECG signal with a high pass filter. The ECG signal may be pre-processed. For example, a high pass filtering of the ECG signal can be used to remove any baseline drift of the ECG signal.

A filter for filtering the ECG signal may, in some examples, combine two essential characteristics. It may achieve both noise reduction of the ECG signal and signal preservation. Digital filters indicated above may fulfil these requirements. For example, a digital median filter is a non-linear filter or is a rank-selection filter.

Filtering of the ECG signal, for example, a high pass filtering, can be used for filtering the overall ECG signal or may be applied to a portion of the ECG signal. Further, a single filter may be used for filtering the overall ECG signal. But it is also possible to apply various filters to different portions of the ECG signal. This means that the ECG signal which goes into an QT algorithm may be pre-processed as described above to remove any baseline drift or signal offset.

The high pass filter can have a cut-off frequency <1 Hz, preferred <0.2 Hz, more preferred 0.05 Hz, and most preferred 0.02 Hz.

The methods for determining a QT interval of an ECG signal may further comprise the step of determining a time window of the ECG signal used for determining a median isoelectric line. The median isoelectric line is also called isoelectric baseline.

In a first step, a period or a time window of the ECG signal used for filtering may be determined or fixed. Then the ECG signal is filtered within the selected time window. The ECG signal filtered within the selected period or time window may be used for determining a median isoelectric line or an isoelectric baseline. Further, a voltage difference of the median isoelectric line with respect to the zero line may be calculated. Based on the result, an item of the above list may be selected for determining the endpoint of the QT interval. Further, the determined voltage difference may be used for correcting the determined end of the T wave.

A combination of various filtering or window techniques to determine the offset of the ECG signal can help to accurately remove the signal baseline drift, and thereby providing a zero offset ECG signal of reference line expected by an algorithm applied for fixing an endpoint of the T wave of the ECG signal.

The time window of the ECG signal used for determining the median isoelectric line may comprise at least one of: using a time window of the ECG signal prior to a P wave and using a time window of the ECG signal in a PR segment. Typically, these segments of the ECG signal are essentially flat. Therefore, these segments are very well suited for determining a difference between the isoelectric line and the zero line of a QT measurement system. For example, the difference can be used for respectively correcting the determination of the endpoint of the QT interval. The difference may also be used for selecting one or more items of the above list for fixing the endpoint of the T wave.

The methods for determining the QT interval of an ECG signal can further comprise the step of determining a heart frequency from the ECG signal, and/or correcting the QT interval for the heart frequency. For example, the determined QT interval can be corrected for the heart frequency using the formula of Fridericia.

The methods for determining the QT interval of an ECG signal can further comprise the step of not determining the QT interval, if the heart frequency exceeds a predetermined number of beats per minute. Thus, the QT measurement or the determining of the QT interval is less prone to errors, and no or less incorrect QT values are provided.

The mentioned step of not determining the QT interval is particularly suitable if the QT interval is measured periodically and/or automatically, as mentioned above.

The predetermined number of beats per minute can be <140, preferably <125, more preferred <110, and most preferred <100.

The methods for determining the QT interval of an ECG signal can further comprise the step of outputting an alert if a duration of the QT interval exceeds a predetermined threshold. The alert can comprise the determined QT interval and the heart frequency.

The methods for determining the QT interval of an ECG signal can further comprise the step of outputting an alert if a duration of the QTc interval exceeds a predetermined threshold. The alert can comprise the determined QTc interval. The methods can minimize the data transmission necessary for a long-term monitoring of a patient.

The methods for determining the QT interval of an ECG signal can further comprise the step of outputting an alert if a duration of the QT interval and/or QTc interval does not reach a predetermined threshold. The alert can comprise the determined QT and/or QTc interval.

The methods for determining the QT interval of an ECG signal can further comprise the step of obtaining the ECG signal from a sensor connected to an implantable cardiac monitor (ICM).

The methods for determining the QT interval of an ECG signal can additionally comprise the step of setting a noise event marker if a noise level of the ECG signal exceeds a predetermined level. This approach secures that QT intervals are only determined if their respective error intervals are low.

The methods for determining the QT interval of an ECG signal can further comprise the step of not determining the QT interval if a noise event marker is detected in the cardiac cycle.

The methods as described above may be used for evaluating ECG signals in real time and/or they can be used for evaluating stored ECG signals of a long-term tracking process.

The methods as described herein may be used for acquired (real-time) QT data and/or long-term tracking of QT data.

According to an exemplary embodiment, a computer program product has instructions for causing a computer to execute any of the method steps of the aspects described herein.

According to another exemplary embodiment, a non-transitory computer-readable medium comprises instructions stored thereon, that when executed on a computer, perform the method steps of one of the aspects described herein.

According to a further exemplary embodiment, an apparatus for monitoring a QT interval of an electrocardiogram (ECG) signal comprises: a processor operable to: (a) determine a maximum slope of the ECG signal after a T wave maximum of the ECG signal; and b. fix an end of the QT interval at least in part based on (and/or by) performing at least one of: extrapolate the maximum slope to a zero line of the ECG signal;
determine a tine position of the ECG signal after the T wave maximum at which the slope is for the first time within a range of 70% to 20%, preferred 66% to 25% of the maximum slope;
determine a time position of the ECG signal at which an absolute value of a difference of the ECG signal and a product of the maximum slope multiplied by a length of a time interval which is used for determining the maximum slope is below a predetermined threshold;
calculate a second derivative of the ECG signal to determine an inflection point which is closest to the time position of the maximum slope.

According to a further exemplary embodiment, an apparatus for monitoring a QT interval of an electrocardiogram (ECG) signal may comprise: a processor operable to determine a maximum slope of the ECG signal after a T wave maximum of the ECG signal to extrapolate the maximum slope to a zero line of the ECG signal for fixing an end of the QT interval.

According to a further exemplary embodiment, an apparatus for monitoring a QT interval of an electrocardiogram (ECG) signal comprises: a processor operable to: determine a maximum slope of the ECG signal after a T wave maximum of the ECG signal; and extrapolate the maximum slope to zero line of the ECG signal for fixing an end of the QT interval.

According to a further exemplary embodiment, an apparatus for monitoring a QT interval of an electrocardiogram (ECG) signal comprises: a processor operable to search for a zero slope of the ECG signal after a T wave maximum of the ECG signal for fixing an end of the QT interval of the ECG signal. In some examples, the zero slope may be a first zero slope, and the processor may further be operable to determine a time of the first zero slope for fixing an end of the QT interval of the ECG signal.

According to another exemplary embodiment, an apparatus for monitoring a QT interval of an electrocardiogram (ECG) signal comprises: a processor operable to: (a) search for a zero slope of the ECG signal after a T wave maximum of the ECG signal; and (b) fix an end of the QT interval of the ECG signal at least in part based on determining a time position of a first zero slope of the ECG signal.

The processors described herein may further be operable to use at least one of: a high pass filter, a wavelet filter, and a median filter to perform filtering of the ECG signal.

The apparatuses may have a non-volatile memory storing one or more algorithms for performing one or more method steps of the above defined aspects. The one or more algorithms can be executed by the processor of the apparatuses. The apparatuses may have a volatile memory for storing and/or buffering the ECG signal.

The apparatuses can have at least one sensor operable to detect the ECG signal. The at least one sensor may be attached to a body of a patient and/or may be inserted into the body of the patient. The apparatuses may communicate wire based and/or wirelessly with the at least one sensor.

The apparatuses can comprise a transceiver operable to receive data from a remote transceiver station and transmit data to the remote transceiver station. The transceiver may communicate wirelessly and/or wire based with the remote transceiver station.

The apparatuses may be an implantable cardiac monitor (ICM).

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

In order to better understand the present disclosure and to appreciate its practical applications, the following figures are provided and referenced hereafter. It should be noted that the figures are given as examples only and in no way limit the scope of the disclosure.

FIG. 1 schematically shows an example of an electrocardiogram (ECG) signal and an example of an apparatus which can detect the ECG signal;

FIG. 2 presents an example of a measured ECG signal and graphically illustrates two methods of determining an end or an endpoint of a QT interval;

FIG. 3 shows a further example of an ECG signal and two portions or time windows which can be used for filtering the ECG signal for determining a median isoelectric line;

FIG. 4 presents again the ECG signal of FIG. 2 and graphically illustrates two further items for determining an endpoint of the QT interval;

FIG. 5 illustrates the determination of the end of the T wave of an ECG signal by using the second derivative of the ECG signal;

FIG. 6 schematically illustrates a method for determined an end of a QT interval based on a maximum slope detection after the T wave maximum;

FIG. 7 represents a flow diagram of a method used for determining a QT interval of an ECG signal;

FIG. 8 shows a flow diagram of a method for determining a QT interval of an ECG signal;

FIG. 9 gives a flow diagram of a method for determining a QT interval of an ECG signal; and

FIG. 10 shows a flow diagram of a method for determining a QT interval of an ECG signal.

### DETAILED DESCRIPTION

In the following, the present disclosure will be more fully described hereinafter with reference to the accompanying figures, in which exemplary embodiments are illustrated. However, the present invention may be embodied in different forms and should not be construed as limited to the embodiments set forth herein. Rather, these embodiments are provided so that this disclosure will be thorough and will convey the scope of the present invention to persons skilled in the art.

The present disclosure describes methods and apparatuses for automatically detecting conditions related to the QT interval as outlined above. An electrocardiogram (ECG) signal is used to gather some information associated with some aspects of a cardiac cycle, as, for example, a start of the Q wave, an end or endpoint of the T wave, and a time interval between R wave peaks of successive cardiac cycles. The calculated QT interval can be analysed to detect conditions related to QT interval variations, in particular QT interval extensions, containing intermittent events as well as gradual changes of the QT interval.

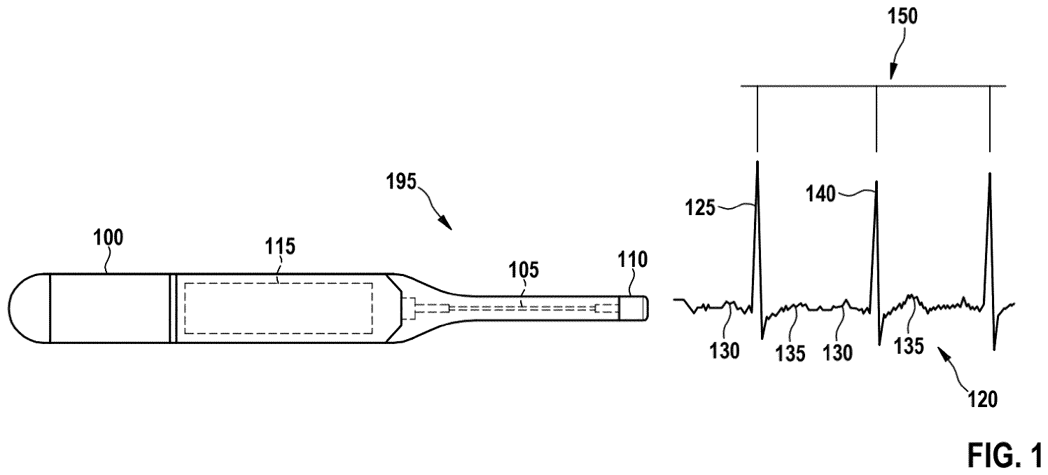

FIG. 1 schematically illustrates an implantable or insertable cardiac monitor (ICM) 100 which is an example of an apparatus 100 on the left part of the diagram 195. The ICM 100 comprises a sensor 110 which is connected to the ICM 100 via a cable 105, for example. Alternatively, the sensor 110 may wirelessly be connected to the ICM 100. The sensor 110 can detect an ECG signal 120 and the cable 105 transmits the detected ECG signal 120 to the ICM 100. Both, the cable 105 and the sensor 110 can be designed to be implantable into a body of a patient. The sensor 110 can be located at a position in the body of a patient so that it can reliably detect the ECG signal 120 of the patient. It can be a benefit of an implanted sensor 110 that it can be fixedly positioned and/or that it can reliably measure ECG signals 120 over a long period.

The right part of the diagram 195 presents two cycles of an ECG signal 120. The ECG signal 120 contains a P wave 130, a QRS complex 125 and a T wave 135. The heartbeat frequency can be calculated based on the time interval between two consecutive R wave peaks 140. The right part of the diagram 195 also depicts durations of beats 150 of the ECG signal 120.

The ICM 100 contains a processor 115 which can process the ECG signal 120 detected by the sensor 110. The processor 115 may, for example, be a microprocessor. The ICM 100 may include a volatile and/or a non-volatile memory (not shown in diagram 195). The volatile memory may store or buffer the detected ECG signal 120. The non-volatile memory may store one or more algorithms to detect and/or to process the ECG signal 120. Further, the non-volatile memory may store one or more algorithms realizing one or more digital filters which can be used for filtering a portion or the overall ECG signal 120. The processor 115 may perform both types of algorithms, i.e., processing and filtering algorithms. Moreover, one or more analogue filters may be used for filtering the ECG signal 120 (not represented in FIG. 1.

Alternatively, or additionally, the ICM 100 may contain a processor specifically designed for performing digital filtering of the ECG signal 120 (not shown in FIG. 1). The specific processor may comprise an ASIC (Application Specific Integrated Circuit) and/or a FPGA (Field Programmable Gate Array). For example, the specific processor may implement one or more digital filters. The digital filters may be implemented in hardware, software or a combination thereof. The digital filter may comprise a low pass filter, a wavelet filter and/or a median filter. The ICM 100 can comprise a battery (not shown).

The methods defined according to the present disclosure are not limited to be performed by the ICM 100. They can also be executed by a monitoring device attached to the body of a patient and/or by a separate device. Further, for example, these methods may also be performed by a cardiac pacemaker.

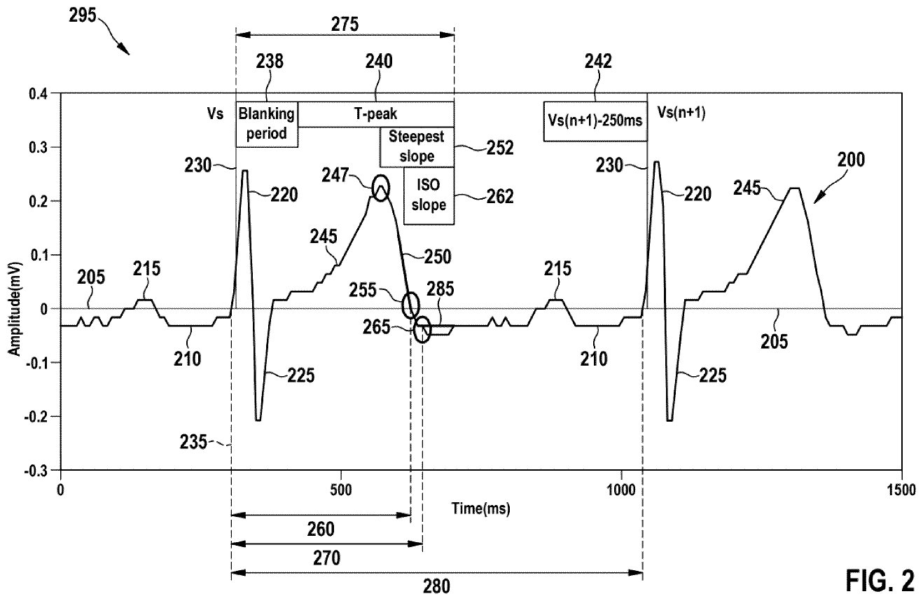

FIG. 2 presents an ECG signal 200 measured by the sensor 110 of the ICM 100 in greater detail. The diagram 295 of FIG. 2 again shows two cardiac cycles of the ECG signal 200. The diagram 295 is used to discuss two of the disclosed methods for determining a QT interval of the ECG signal 200.

The zero-voltage line 205 is a line having a zero-voltage amplitude of the ECG signal 200. The isoelectric line 210 of the ECG signal 200 is the line having a zero slope and which is close to but typically not identical to the zero-voltage line 205. The isoelectric line 210 is the reference potential of the sensor 110 of the ICM 100. The P waves of the ECG signal 200 are denoted with the reference sign 215. The peaks 220 represent the QRS complex 225 of the ECG signal 200.

The vertical lines 230 illustrate an example of a sense event marker Vs 230. The sense event marker 230 detects and/or marks the beginning or start of the QRS complex 225 by an autosense algorithm once the ECG signal 200 crosses an active sensing threshold. The autosense algorithm may also generate one or several noise event markers Vn (not shown in FIG. 2). The methods described in detail in the following discard any Vs marker 230 that are followed by a Vn marker to obtain intervals between two Vs markers 230 that are free from Vn markers.

After crossing the active sensing threshold, a detection hold-off period (DHP) is started (not represented in diagram 295). During the DHP period no further detection events can be generated which effectively limits the maximum detection rate of the QRS complex 225. Further, a peak detection window (PDW) can also be started after detecting the sense event marker 230. During the PDW, the signal amplitude of the ECG signal 200 is tracked by a threshold reference (TR) register. After the DHP expires, a new sensing threshold can be derived from the peak measurement as a percentage of the TR, and the sensing threshold continues to decrease. This threshold countdown continues until the sensing threshold reaches a target threshold.

Irrespective of the method used for fixing the endpoint of the QT interval, the start of the Q wave 235 can be determined as a fixed offset to the sense event marker 230. Additionally, and/or alternatively, the Q wave start 235 can be obtained as a deviation of a predetermined threshold from the isoelectric line 210 in a PQ segment of the ECG signal 200. The PQ segment is the interval between the endpoint of the P wave 215 and the begin of the QRS complex 225 which corresponds or is close to the Q wave start 235. Further, the start of the Q wave 235 can also be obtained by detecting a slope of the ECG signal 200 exceeding the isoelectric line 210 by a predetermined threshold in the PQ segment.

Detection of the sense event marker 230 can also start a blanking period 238. The blanking period 238 typically has a time interval which corresponds to the combined duration of the QRS complex 225 and the ST segment. On expiration of the blanking period 238, a T wave maximum search interval 240 starts and can continue for a duration of 600 ms or is stopped 250 ms prior the next sense event marker 230. The T wave 245 is analysed within the T wave maximum search interval 240 by searching the largest absolute value of the ECG signal 200 in order to determine the peak 247 of the T wave 245. The duration of the blanking period 238 and the T wave maximum search interval 240 denoted in the diagram 295 by the reference numeral 275 is smaller than the interval between two subsequent sense event markers Vs 230.

In the next step, the steepest slope of the T wave 245 is calculated after the peak 247 of the T wave 245 is crossed. The maximum slope 250 is searched in the interval 252. The interval 252 begins at the (time) position of the T wave peak 247 and ends at the end of the T wave maximum search interval 240. The time duration used to calculate the slope can be critical and a wide range is tested. Best results are obtained with a time window of approximately 40 ms. Further, about 20 samples are averaged at a sampling frequency of 512 Hz.

Then, in a first method, the maximum slope 250 is extrapolated to meet the zero-voltage line 205 of the ECG signal 200. The end 255 or the endpoint 255 of the T wave 245 which corresponds to the end of the QT interval is taken the intersection point 255 of the maximum slope 250 with the zero-voltage line 205 of the ECG signal 200. The QT interval 260 is or may be calculated as the difference of the endpoint 255 and the Q wave start 235.

As can be recognized from the diagram 295, a different endpoint of the QT interval is detected if the maximum slope 250 is extrapolated to the isoelectric line 210 instead of the zero line 205. The extrapolated maximum slope intersects the isoelectric line 210 at a position which is in between the endpoints 255 and 265. Thus, as discussed above, an offset and/or a drift of the isoelectric line 210 may have a significant effect on the determination of the endpoint 255 of the QT interval 260. It is therefore necessary to precisely know the offset of the isoelectric line 210 with respect to the zero line 205 of the ICM 100. For accurately establishing the voltage of the isoelectric line 210, one or several intervals or time windows of the ECG signal 200 have to be identified which can be used for analysing the position of the isoelectric line 210 with respect to the zero line 205.

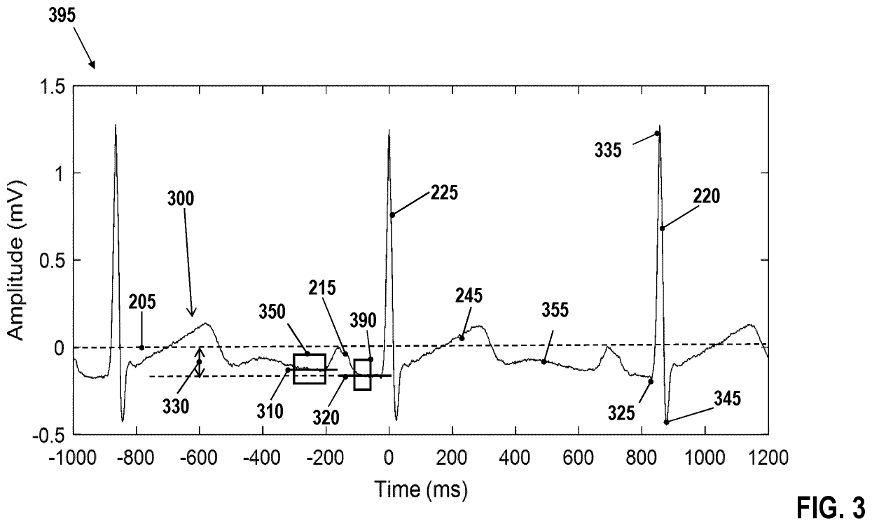

Diagram 395 of FIG. 3 presents again two cardiac cycles. As can be seen from FIG. 3, the ECG signal 300 has an offset with respect to the zero line 205. By definition, the isoelectric line 210 has a zero slope. Typically, there are two periods or time windows in which an ECG signal 120, 200, 300 shows an essentially flat characteristic. A first window 350 or a first period 350 is located before the P wave 215 and a second window 390 or second period 390 is located in front of the QRS complex 225. These windows 350 and 390 can be used for the determination of the voltage level of the isoelectric line 210. Further, FIG. 3 additionally indicates all essential waves of a cardiac cycle. After the P wave 215, the QRS complex 225 contains the Q wave 325, the R wave 335, and the S wave 345. The QRS complex 225 of the ECG signal 200 is followed by the T wave 245 and a small U wave 355.

When the position and/or the length of the window 350 and or the window 390 are fixed, the ECG signal 120, 200, 300 can be filtered in this/these time windows 350, 390. Typically, one or more digital filters are applied for filtering the ECG signal 120, 200, 300 within one or both time windows 350 and 390. As digital filters one or more low pass filters, one or more wavelet filters, or one or more median filters may be applied. A median isoelectric line 310, 320 or an isoelectric baseline 310, 320 can be determined by filtering the ECG signal 120, 200, 300 within the selected time window(s) 350, 390.

The median isoelectric line 310, 320 may be used for correcting an offset and/or a drift of the ECG signal 120, 200, 300. If this is done the intersection points of the extrapolated maximum slope 250 with the zero-line 205 and the median isoelectric baseline 310, 320 coincides. Thus, both procedures or algorithms for fixing the endpoint 255 lead to an identical QT interval length 260.

Based on the difference 330 between the median isoelectric baseline 310, 320 and the zero line 205, it is also possible to decide which item or which procedure is best suited for reliably determining the length of the QT interval.

Again, with reference to FIG. 2, a second method may be used for determining an endpoint of the T wave 245 of the ECG signal 200. The second method uses a first begin of the isoelectric line 210, the median isoelectric line 320 or the isoelectric baseline 320 after the T wave 245 to fix the endpoint 265 of the T wave 245. In order to determine the starting point 265 of the isoelectric line 210 after the T wave 245, a zero slope 285 is searched for in the interval 262 starting at the point of the maximum slope 250 and ending at the endpoint of the T wave maximum search interval 240. As discussed in the context of FIG. 3, the isoelectric line 210 has a zero slope 285.

As is illustrated in the diagram 295, the end point 265 based on the zero-slope determination is slightly right of the intersection point 255. This means that the QT interval 270, determined as the difference of the endpoint 265 and the start of the Q wave 235, is slightly larger than the QT interval 260 determined when using the first method discussed above. Thus, the second method may not always correctly detect the end of the T wave 245 but may lead to a delayed detection of the endpoint 265 resulting in an enlarged QT interval 270.

The length difference of the QT intervals 260 and 270 is caused to a large extent by the difference 330 or offset 330 between the zero line 205 of the ECG signal 200 and the isoelectric line 210 or the median isoelectric line 320. The measured time difference between the QT intervals 260 and 270 significantly reduces when the zero line 205 and the isoelectric line 210 or the median isoelectric line 320 match. However, the difference 330 increases when the ECG signal 120, 200 300 drifts away from the zero line 205 of the ICM 100.

Based on the difference 330 between the median isoelectric line 320 and the zero line 205, it is also possible to decide whether to use the first or the second method for calculating the end 255, 265 of the QT interval 260, 270.

It is of course possible to combine both methods and use the second method as a refinement of the first one or vice versa. For example, instead of extrapolating the maximum slope 250 to meet the zero-voltage line 205 of the ECG signal 200, additionally or alternatively, it can be extrapolated to meet the isoelectric line 210, e.g., as determined using the zero slope aspects described herein. Furthermore, several of the methods described herein can be used to calculate the two QT intervals 260 and 270 and take an average or a weighted average as the QT interval.

It is also possible to adapt the method for fixing the beginning 230 of the QT interval 260, 270 to the method selected for determining the endpoint 255, 265 of the QT interval 260, 270. Thus, the accuracy of the QT interval length determination may be augmented.

Further, the time interval between two consecutive sense event markers Vs(n) 230 and Vs(n+1) 230 can be used to calculate the heart frequency of the cardiac cycle 280 for which the QT interval 260, 270 is determined. It is beneficial to use Vs-Vs intervals that are larger than 600 ms in duration (which corresponds to a heart rate below 100 bpm (beats per minute) for calculating the endpoints 255 and/or 265. Finally, the QT interval 260, 270 can be corrected with the heart frequency determined from the Vs-Vs interval 280. For example, the correction can be done by using the formula of Fridericia.

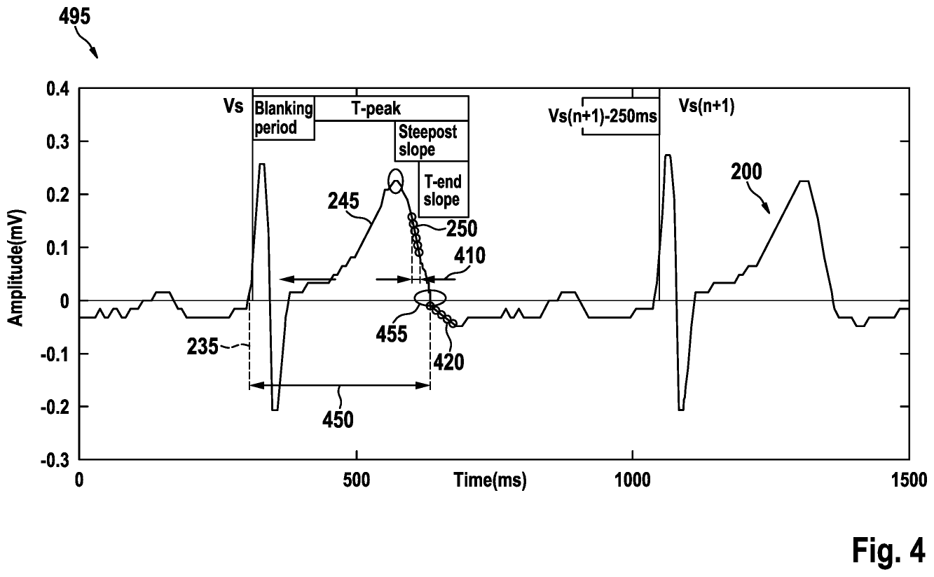

Diagram 495 of FIG. 4 schematically presents further items of the list indicated above for calculating the endpoint 455 of a QT interval 450. FIG. 4 again shows the ECG signal 200 of FIG. 2. In the interval 252 of FIG. 2 (shown as “Steepest Slope” in FIG. 4), the maximum slope 250 of the T wave 245 is represented. Additionally, FIG. 4 schematically illustrates the length of the time window 410 which is used for determining the maximum slope 250. The duration of the interval 410 or the time window 410 may vary within a range of a few milliseconds up close to about 100 milliseconds depending on the specific form of the T wave 245 of the ECG signal 120, 200, 300. Typically, the time window 410 extends from about 20 ms to approximately 40 ms. For the current algorithm a value of 10 to 50 ms, 20 to 40 ms or about 31 ms (16 samples at 512 Hz sampling frequency) was used. The algorithm is able to keep track of the slope value and the slope polarity.

In in further approach, the end 455 of the T wave 245 T_{end}(tₙ) may be determined by minimizing the absolute value of the difference between the current ECG signal S_{E}(tₙ) and the maximum slope ΔS_{E,max}(tₖ) multiplied by the length Δt of the time window 410 used for fixing the maximum slope. The following equation indicates the calculation of the T wave end 457:

T_{end}(tₙ)=|S_{E}(tₙ)−ΔS_{E,max}(tₖ)·Δt|

wherein Δt denotes the length of a time window 410. Instead of using the smallest difference as the end 455 or the endpoint 455 of the T wave 245, it is also possible to predetermine a threshold which identifies the end 455 of the T wave as the time when T_{end}(tₙ) is below a predetermined threshold. The endpoint search may be limited to an interval exemplarily indicated as “T-end slope” in FIG. 4.

A further algorithm for determining the endpoint 455 of the T wave 245 is based on the calculation of the second derivative of the ECG signal 120, 200, 300 in a period starting at the time of the maximum slope 250 and ending at the end of the T wave maximum search interval 240 (indicated as “T-peak” in FIG. 4).

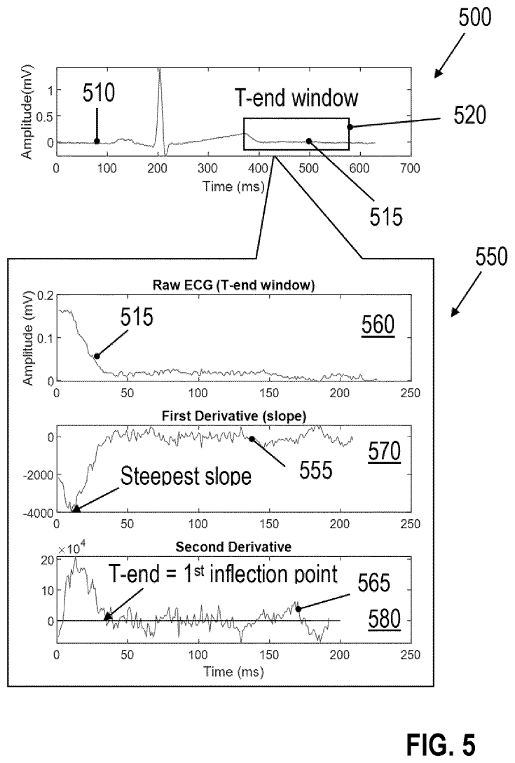

This approach is illustrated in FIG. 5.

The diagram 500 of FIG. 5 shows an ECG signal 510 used for illustrating the second derivative approach. A T end window 520 symbolizes the time window into with the second derivative of the ECG signal 515 is formed. It begins at the time position of the T wave maximum 247 and ends at the end of the T wave peak search interval 240. The diagram 550 of FIG. 5 presents an enlarged section of the ECG signal 515 in the T end window 520. The upper partial image 560 of the diagram 550 presents the ECG signal 515 within the T end window 520. The middle partial image 570 of the diagram 550 represents the first derivative 555 of the ECG signal 515. The maximum slope 250 of the T wave 245 after the T wave maximum 247 is indicated by an arrow in the partial image 570.

The lower partial image 580 of the diagram 550 of FIG. 5 presents the second derivative 565 of the ECG signal 515 calculated in the T end window 520. The first inflection point (time position at which the second derivative is zero) is indicated by an arrow. The end of the T wave 245 corresponds the first inflection point after the maximum slope 250 of the T wave 247. This means that the inflection point which is closest to the maximum slope 250 denotes the end 455 of the T wave 245.

Moreover, another approach or another algorithm may detect the end 455 of the T wave 245 when the current or instantaneous slope of the ECG signal 120, 200, 300 is for the first time within a predetermined slope range in the interval beginning at the time position of the maximum slope 250 and ending at the end of the interval 252. The approach is illustrated in the partial image 570 of the diagram 550 of FIG. 5. As discussed above, after the time position of maximum slope 250, the first derivative 555 of the ECG signal 120, 200, 300, 510 reduces and finally reaches a zero at a voltage level of the isoelectric line 210. This slope range may be specified depending on the specific form of the T wave 245 of the ECG signal 120, 200, 300, 510. The predetermined slope range may be referred to the maximum slope 250. The preferred slope range for determining the end 455 of the T wave 245 may be within two thirds to one fourth of the maximum slope 250. As already discussed in the context of FIG. 2, the length of the QT interval 450 may be determined as the difference between the start 235 of the Q wave 325 and the endpoint 455 of the T wave 245.

The last three procedures or approaches discussed in the context of FIGS. 4 and 5 have the advantage that they do not refer to the zero line 205, the isoelectric line 210 or the median isoelectric line 310, 320. Further, two or more of the discussed approaches or algorithms can be used in combination depending on the specific form of the ECG signal 120, 200, 300 to improve the accuracy of the determination of the length of the QT interval 450. Moreover, the first methods and the second method, or one or more of the algorithms of the first method and the algorithm of the second method for determining the endpoint 255, 265, 455 can be combined for increasing the accuracy of the determination of the QT interval 260, 270, 450.

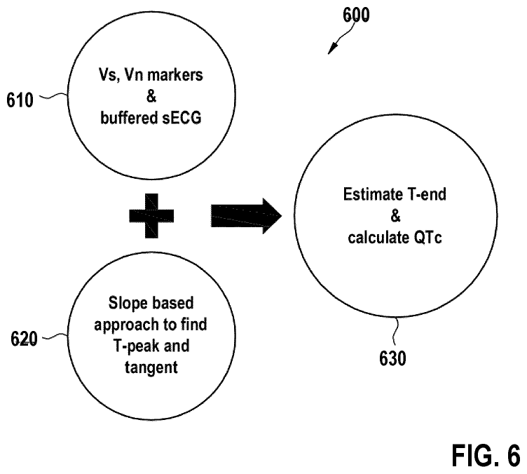

The diagram 600 of FIG. 6 summarizes the methods presented in this application for determining the length of the QT interval 260, 270, 450 of an ECG signal 120, 200, 300. At step 310, sense event markers Vs 230 and noise event markers Vn are generated and stored or buffered in a memory of the ICM 100. The sense (Vs) and the noise event marker (Vn) obtained by using an autosense algorithm are used to identify clean individual QRS complexes 225 and/or cardiac cycles to calculate the QT interval 260, 270, 450. The autosense algorithm is used irrespective of the method or algorithm applied to determine the end 255, 265, 455 of the T wave 245. Additionally, the ECG signal 120, 200, 300 may be pre-processed by the processor 115 of the ICM 100, for example, to eliminate any drift of the electrostatic line 210 or the median electrostatic line 310, 320 of the ECG signal 120, 200, 300. A drift and/or an offset 330 may be removed by filtering the ECG signal 120, 200, 300, for example, within the time window 310, 320. The filtering may be performed by using a high pass filter (not shown in FIG. 6). Preferably one or more digital filters are used.

At step 620, a slope-based approach may be used to calculate the peak 247 of the T wave 245 of the ECG signal 200. Then the maximum slope 250 of the T wave 245 is calculated in the interval 252. The maximum slope 250 may be extrapolated to the zero line 205. Alternatively, and/or additionally, a zero slope of the ECG signal 120, 200, 300 can be determined in the interval 262.

Further, the endpoint 455 of the QT interval 450 may be determined by minimizing the absolute value of the difference of the current or instantaneous ECG signal 120, 200, 300 and the maximum slope 250 multiplied by the slope length, i.e., the time window 410 used for fixing the maximum slope 250. Moreover, the endpoint 455 of the QT interval 450 may be fixed by calculating the second derivative of the ECG signal 120, 200, 300 in the T wave maximum search interval 240 beyond the time position of maximum slope 250 and searching for the deflection point in this interval which is closest to the maximum slope 250. Finally, the endpoint 450 of the QT interval 450 may fixed as a certain percentage of the maximum slope 250. For example, the end 455 of the T-wave 245 may be defined as the time position when the current slope is in a range of two thirds to one fourth of the maximum slope 250. A window of data between two consecutive Vs markers without any Vn marker is chosen for fixing the endpoints 255 and/or 265.

Then at step 630, the QT interval 260, 270, 450 is calculated from the intersection point 255, 455 and/or the time position when a zero slope 285 is detected for the first time after the maximum slope 250 of the T wave 245 is passed. This time corresponds to the starting point 265 of the isoelectric line 210 or the isoelectric baseline 310, 320 after the T wave 245. The QT interval 260, 270, 450 and the corresponding heart rate determined from the Vs-Vs interval 280 can be sent to a remote monitoring unit (not shown in FIG. 6) for further processing, for instance, like calculating a QTc interval. Alternatively, the QTc interval may be calculated by the processor 115 of the ICM 100 using the Vs-Vs duration 280 reflecting the heart frequency. Finally, an alert may be sent to a care provider team through remote monitoring when the QTc interval is greater than a predetermine threshold (not shown in FIG. 6).

Using the ICM 100 to manage risks associated with QTc changes provides a daily or on-demand determination of the QT interval 260, 270, 450 which would otherwise require an office visit and a specialty appointment.

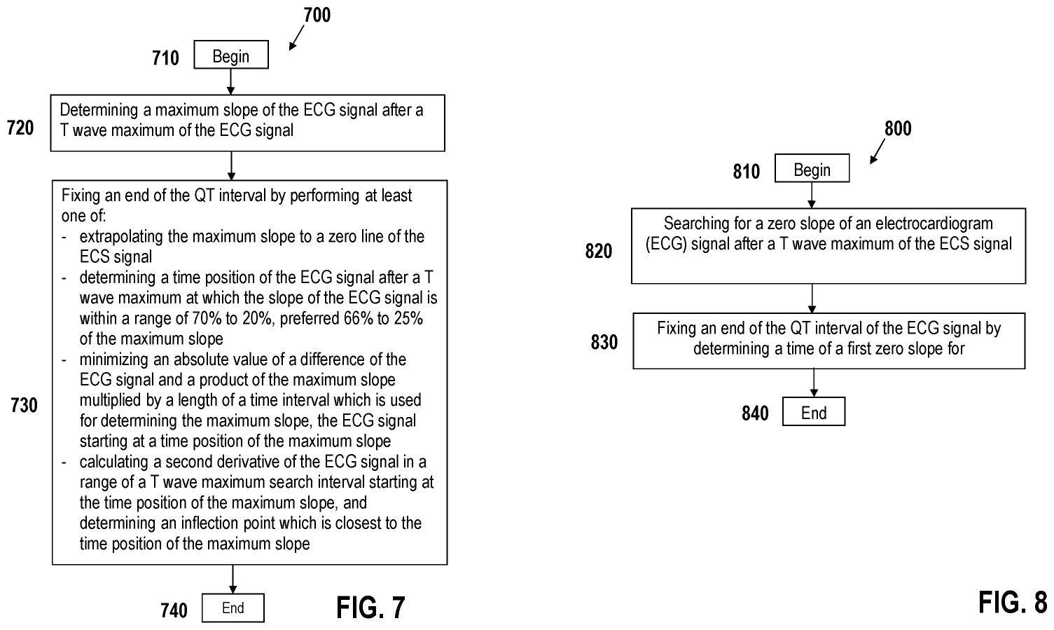

FIG. 7 shows a flow chart 700 of a first method for determining a QT interval 260 and 270 of an ECG signal 120, 200, 300. The method begins at 710. As a first step 720, a maximum slope 250 of the ECG signal 120, 200, 300 after a T wave maximum 247 of the ECG signal 129, 200, 300 is determined. This step can be performed by an algorithm executed by the processor 115 of the ICM 100.

At step 730 an end 255, 455 of the QT interval 260, 450 is fixed by performing at least one of:
extrapolating the maximum slope 250 to a zero line 205 of the ECG signal 120, 200, 300;
determining a time position of the ECG signal 120, 200, 300 after the T wave maximum 247 at which the slope is within a range of 70% to 20%, preferred 66% to 25% of the maximum slope 250;
minimizing an absolute value of a difference of the ECG signal 120, 200, 300 and a product of the maximum slope 250 multiplied by a length of a time interval 410 which is used for determining the maximum slope 250, the ECG signal 120, 200, 300 starting at a time position of the maximum slope 250;
calculating a second derivative of the ECG signal 120, 200, 300 in a range of a T wave maximum search interval 240 starting at a time position of the maximum slope 250, and determining an inflection point which is closest to the time position of the maximum slope 250.

For example, this step can also be performed by an algorithm executed by the processor 115 of the ICM 100. The method ends at 740.

FIG. 8 presents a flow diagram 700 of a second method for determining a QT interval 270 of an ECG signal 120, 200 300. The method begins at 810. At step 820, a first zero slope 285 of the ECG signal 120, 200, 400 after a T wave 245 maximum 247 of the ECG signal 120, 200, 300 is searched for. This step can be performed by an algorithm executed by the processor 115 of the ICM 100. At step 830, an end 265 of the QT interval 270 is fixed by determining a time position of a first zero slope 285 of the ECG signal 120, 200, 300. This step may also be performed by an algorithm executed by the processor 115 of the ICM 100. The method ends at step 840.

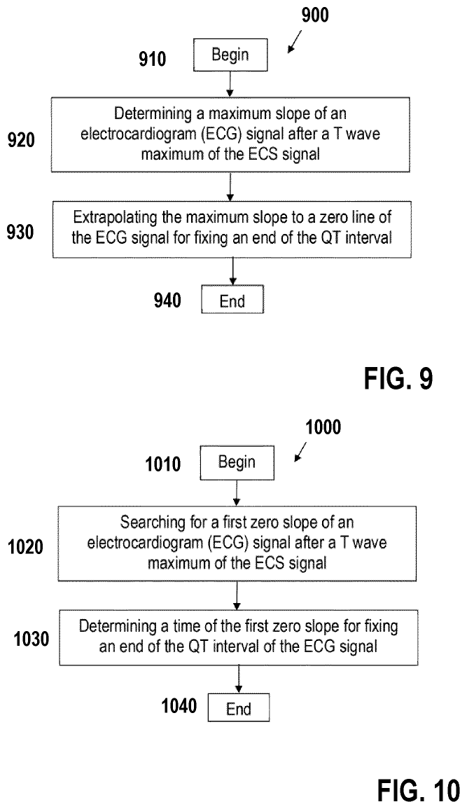

FIG. 9 shows a flow chart 900 of a first method for determining a QT interval 260 of an ECG signal 120, 200. The method begins at 910. At step 920, a maximum slope 250 of the ECG signal 120, 200 after a T wave 245 maximum 247 of the ECG signal 120, 200 is determined. This step can be performed by an algorithm executed by the processor 115 of the ICM 100. At step 930, the maximum slope 250 is extrapolated to a zero voltage line 205 and/or to an isoelectric line of the ECG signal 120, 200 for fixing and end 255 of the QT interval 260. This step may be performed by an algorithm executed by the processor 115 of the ICM 100. The method ends at step 940.

Finally, FIG. 10 presents a flow diagram 500 of a second method for determining a QT interval 270 of an ECG signal 120, 200. The method begins at 1010. At step 1020, a first zero slope 285 of the ECG signal 120, 200 after a T wave 245 maximum 247 of the ECG signal 120, 200 is searched for. This step can be performed by an algorithm executed by the processor 115 of the ICM 100. At step 1030, a time of the first zero slope 285 is determined for fixing an end 265 of the QT interval 270 of the ECG signal 120, 200. This step may also be performed by an algorithm executed by the processor 115 of the ICM 100. The method ends at step 1040.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

In the following, further embodiments are described to facilitate the understanding of the present invention:

1. A method for determining a QT interval of an electrocardiogram (ECG) signal, the method comprising the steps:
determining a maximum slope of the ECG signal after a T wave maximum of the ECG signal; and
extrapolating the maximum slope to a zero line of the ECG signal for fixing an end of the QT interval.

2. A method for determining a QT interval of an electrocardiogram (ECG) signal, the method comprising:
searching for a zero slope of the ECG signal after a T wave maximum of the ECG signal for fixing an end of the QT interval of the ECG signal.

3. The method of embodiment 1 or 2, further comprising the step of determining a start of a Q wave of the ECG signal.

4. The method of the preceding embodiment, wherein determining the start of the Q wave comprises at least one of determining a sense event marker and determining a QRS complex.

5. The method of the preceding embodiment, wherein the sense event marker detects at least one of a predetermined variation of the zero slope of the ECG signal in a PQ segment and an electrical potential exceeding a predetermined threshold in the PQ segment.

6. The method of any one of the preceding embodiments wherein determining the T wave maximum comprises determining a T wave maximum search interval for detecting the T wave maximum.

7. The method of embodiment 6, wherein the T wave maximum search interval begins at the end of an ST segment of the ECG signal, and/or wherein the T wave maximum search interval has a duration of 150 ms to 600 ms, preferably 200 ms to 500 ms, most preferably 250 ms to 400 ms.

8. The method of any one of embodiments 1 and 3 to 7 referred back to embodiment 1, wherein determining the maximum slope comprises calculating a slope of the ECG signal by averaging the slope within a time interval of 2 ms to 100 ms, preferably 5 ms to 80 ms, more preferred 10 ms to 60 ms, most preferred 20 to 40 ms.

9. The method of any one of the preceding embodiments, further comprising the step of filtering the ECG signal with a high pass filter.

10. The method of any of the preceding embodiments further comprising the step: determining a heart frequency from the ECG signal, and/or correcting the QT interval) for the heart frequency.

11. The method of the preceding embodiment, further comprising the step of not determining the QT interval, if the heart frequency exceeds a predetermined number of beats per minute.

12. The method of any one of the preceding claims, further comprising the step of outputting an alert, if a duration of the QT interval exceeds a predetermined threshold.

13. A computer program product having instructions for causing a computer to execute the steps of the method according one of the embodiments 1 to 12.

14. An apparatus for monitoring a QT interval of an electrocardiogram (ECG) signal comprising:
a processor operable to:
   determine a maximum slope of the ECG signal after a T wave maximum of the ECG signal; and
   extrapolate the maximum slope to a zero line of the ECG signal for fixing an end of the QT interval.

15. An apparatus for monitoring a QT interval of an electrocardiogram signal comprising:
a processor operable to:
   search for a zero slope of the ECG signal after a T wave maximum of the ECG signal to fix an end of the QT interval of the ECG signal.

1. A method for determining a QT interval of an electrocardiogram (ECG) signal, comprising the steps:
determining a maximum slope of the ECG signal after a T wave maximum of the ECG signal; and
fixing an end of the QT interval at least in part based on performing at least one of:
extrapolating the maximum slope to a zero line of the ECG signal;
determining a time position of the ECG signal after the T wave maximum at which the slope of the ECG signal is for the first time within a range of 70% to 20%, preferably 66% to 25%, of the maximum slope;
determining a time position at which an absolute value of a difference of the ECG signal and a product of the maximum slope and a length of a time interval which is used for determining the maximum slope is below a predetermined threshold;
calculating a second derivative of the ECG signal to determine an inflection point after the time position of the maximum slope-which is closest to the time position of the maximum slope.

2. A method for determining a QT interval of an electrocardiogram (ECG) signal, the method comprising the steps:
searching for a zero slope of the ECG signal after a T wave maximum of the ECG signal; and
fixing an end of the QT interval of the ECG signal at least in part based on determining a time position of a first zero slope of the ECG signal.

3. The method of claim 1, wherein determining the QT interval comprises using at least two items of feature b. and averaging at least two results obtained when using the at least two items.

4. The method of claim 1, further comprising the step of: determining a start of a Q wave of the ECG signal.

5. The method of claim 4, wherein determining the start of the Q wave comprises at least one of: determining a sense event marker and determining a QRS complex.

6. The method of claim 1, wherein determining the T wave maximum comprises determining a T wave maximum search interval for detecting the T wave maximum.

7. The method of claim 1, wherein determining the maximum slope comprises calculating a slope of the ECG signal by averaging the slope within a time interval of 2 ms to 100 ms, preferably 5 ms to 80 ms, more preferred 10 ms to 60 ms, most preferred ms to 40 ms.

8. The method of claim 1, further comprising the step of filtering the ECG signal, wherein preferably filtering the ECG signal comprises using at least one of: a high pass filter, a wavelet filter, and a median filter.

9. The method of claim 1, further comprising the step of determining a time window of the ECG signal used for determining a median isoelectric line.

10. The method of claim 9, wherein the time window of the ECG signal used for determining the median isoelectric line comprises at least one of: a time window of the ECG signal prior to a P wave and a time window of the ECG signal in a PR segment.

11. The method of claim 1, further comprising the step of: determining a heart frequency from the ECG signal, and/or correcting the QT interval for the heart frequency.

12. A computer program product having instructions for causing a computer to execute the steps of the method according claim 1.

13. An apparatus for monitoring a QT interval of an electrocardiogram (ECG) signal comprising:
a processor operable to:
determine a maximum slope of the ECG signal after a T wave maximum of the ECG signal; and
fix an end of the QT interval based at least in part on performing at least one of:
extrapolate the maximum slope to a zero line of the ECG signal for fixing an end of the QT interval;
determine a time position of the ECG signal after the T wave maximum at which the slope is for the first time within a range of 70% to 20%, preferred 66% to 25% of the maximum slope;
determine a time position at which an absolute value of a difference of the ECG signal and a product of the maximum slope and a length of a time interval which is used for determining the maximum slope is below a predetermined threshold;
calculate a second derivative of the ECG signal to determine an inflection point which is closest to the time position of the maximum slope.

14. An apparatus for monitoring a QT interval of an electrocardiogram (ECG) signal comprising:
a processor operable to:
search for a zero slope of the ECG signal after a T wave maximum of the ECG signal; and
fix an end of the QT interval of the ECG signal at least in part based on determining a time position of a first zero slope of the ECG signal.

15. The apparatus of claim 13, wherein the processor is further operable to use at least one of: a high pass filter, a wavelet filter, and a median filter to perform filtering of the ECG signal.
